(19) United States
(12) Patent Application Publication
(10) Pub. No.: US 2010/0296974 A1
(43) Pub. Date: Nov. 25, 2010

(54) Apparatus for Producing and Delivering Open Air Factor

(75) Inventor: David Murray MacDonald, Irthington Cumbria (GB)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: MOVING SUN LIMITED, Irthington Cumbria (GB)

(21) Appl. No.: 12/595,374

(22) PCT Filed: Apr. 11, 2008
(86) PCT No.: PCT/GB2008/050252

§ 371 Date: Apr. 30, 2010

(30) Foreign Application Priority Data
Apr. 11, 2007 (GB) 0706977.6

(51) Int. Cl. A61L 9/12

(52) U.S. Cl. 422/123

(57) ABSTRACT

An apparatus for producing and delivering open air factor comprises an air supply, a supply of olefin and an ozone supply, means for introducing olefin into the air supply and means for mixing ozone with the olefin/air mixture, wherein the mixing means is arranged such that ozone is mixed with the olefin/air mixture at a period of time after introduction of olefin into the air supply.

### FIELD OF THE INVENTION

The preset invention relates to an apparatus for producing and delivering open air factor, and in particular an apparatus for disinfecting potentially contaminated air using open air factor.

### BACKGROUND OF THE INVENTION

The use of bactericides to disinfect rooms and surfaces gives rise to increasingly prevalent populations of bacteria that are resistant to antibiotics and as a result difficult to treat. Further, antibiotics are ineffective against viruses. In recent times there has been much concern about the occurrence of hospital acquired infection in patients of Methicillin Resistant Staphylococcus Aureus (MRSA).

In order to reduce the prevalence of MRSA hospitals have introduced disinfection policies directed to reducing the possibility of spreading infection from one patient to another. One part of the programme used by hospitals involves the installation of bactericide dispensers which dispense an alcohol based product. These dispensers are easy for hospital staff and visitors to use as the alcohol evaporates rapidly so there is no requirement for hand drying. However, whilst MRSA is controlled at least in part by alcohol based bactericides other potentially harmful bacteria are not. For example, alcohol is a preferred food source for clostridium difficile. Since the introduction of the aforesaid measures to control MRSA there has been an increase in the occurrence of clostridium difficile.

It is interesting to note that acute bacterial and viral infection usually occur in closed environments and in particular closed environments where individuals are resident for prolonged periods of time, such as hospitals, nursing homes, aeroplanes and cruise ships for example.

Where areas are open to fresh air, the natural systems that exist for the control of pathogen populations (namely, the production of hydroxyl radicals from the decay of atmospheric ozone in the presence of olefins) can function. In any area where fresh air is limited, the population of pathogens can rise. This is especially acute in hospitals, where hospital acquired infections are endemic, affecting up to 10% or more of all patients. Such infections can be acquired in a number of ways and the reduction in concentration of killed pathogens compared with fresh or open air has the added effect of increased immunity. Such immunity is caused by pulmonary inoculation as dead pathogens are absorbed through the alveolar system of the lungs.

The present invention utilizes the natural systems used to control pathogens both in atmosphere and in mammals to avoid the use of bactericides. That system relies on the production of short-lived hydroxyl radicals (OH) that react with the phospho-lipid plasma of the pathogen to induce peroxidation in the pathogen bringing about its death.

The natural system referred to was first discovered in the 1960's by researchers at Porton Down in the United Kingdom and TNO in the Netherlands who were investigating how pathogens died in air. They found that the primary method of control is the release of hydroxyl radicals. They found that pathogens died in air at rates that varied depending on weather, airborne pollutants and wind direction. They demonstrated that there was a factor in the atmosphere that destroyed pathogens and called that the Open Air Factor. It was later established that the Open Air Factor was formed by the action of constituents of the atmosphere with a range of olefins, both synthetic and naturally occurring. Terpinenes were of particular efficacy, terpinenes being associated with the scent of flowers or of pine trees. Dutch research showed that a threshold level of ozone concentration of 80 ppb was required, with the presence of olefins, for the Open Air Factor to become fully effective.

In the research at Porton Down referred to above it was found that the Open Air Factor was markedly reduced in a closed chamber. At the time it was thought that the Open Air Factor was absorbed on the surface of the container. However, it is more likely that the effects of the metal container surfaces were to react with free radicals in preference to the free radicals reacting with cell surfaces, resulting in a reduction in efficacy of the Open Air Factor.

An apparatus for generating hydroxyl radicals is described in the published International patent application number WO 2005/026044.

In one apparatus described in WO 2005/026044 ozone is directed towards an outlet over a wick saturated with an olefin. Whilst this is effective in generating hydroxyl radicals it would be desirable to increase the efficiency of production of hydroxyl radicals. Further, improvements could be made to the device described in WO 2005/026044 to improve its ease of use.

The present application is concerned with improvements to the devices described in WO 2005/026044.

### SUMMARY OF THE INVENTION

The present invention is concerned with the development of a nozzle which is adapted in a number of ways which ensure the efficient production of hydroxyl radicals, provide for simple exchange of consumable elements and the safe use of such devices.

According to one aspect of the invention there is provided an apparatus for producing and emitting hydroxyl radicals, the apparatus including an air supply, a supply of olefin and an ozone supply, means for introducing olefin into the air supply and mixing means for mixing ozone with the olefin/air mixture, wherein the mixing means is arranged such that ozone is mixed with the olefin/air mixture at a period of time after introduction of olefin into the air supply.

Preferably the means for introducing olefin into the air supply comprises a dosing chamber. An outlet of the olefin supply is preferably located in the dosing chamber.

Advantageously, the apparatus includes a wick and the wick forms the olefin supply, and preferably the outlet of the olefin supply is an exposed part of the wick.

Ozone is preferably delivered to an ozone mixing chamber and the output of the dosing chamber debouches into the ozone mixing chamber allowing the olefin dosed air to mix with ozone.

Air and ozone are delivered to the dosing and ozone mixing chambers respectively via separate air delivery and ozone delivery ducts, each duct being connected to a supply of air and ozone respectively. In a preferred embodiment, the air delivery duct includes a port which is in fluid communication with the ozone mixing chamber. The port allows a part of the air incoming through the duct to pass through the port directly into the ozone mixing chamber with the remainder of the air passing through the dosing chamber.

Advantageously, the ozone mixing chamber is in the form of a gallery extending radially around the dosing chamber. It is preferred that ozone is introduced to one side of the gallery and air is introduced to the ozone mixing chamber via the port to the other side of the gallery, such that the ozone and the air are traveling in opposite directions. Preferably, the dosing chamber outlet debouches into the gallery to the side on which ozone is introduced, and more preferably, the olefin/air mixture debouches into the gallery in a direction which is tangential to the direction of travel of the ozone.

By delivering a small amount of air in a direction opposite to the direction of flow of the ozone, the time taken for ozone to pass through the ozone mixing chamber, which may comprise the aforesaid gallery, is increased, and as such the time for mixing of ozone with the olefin/air mixture is increased correspondingly. Further where the streams of air and ozone/air/olefin mixture meet turbulence is created which further enhances the mixing effect.

The ozone mixing chamber comprises at least one outlet, which is preferably located where the streams of air and ozone/air/olefin converge.

The dosing chamber, ozone mixing chamber, air and ozone delivery ducts are preferably comprised in a dosing head. The dosing head may be provided with at least one surface which co-operates with at least one surface of another part of the apparatus, co-operation of the said surfaces ensuring the correct position of the dosing head in the apparatus. It follows that any components of the apparatus connected to the dosing head would also be positioned correctly.

It is preferred that the exposed part of the wick is held in a clamp and that the clamp is mounted in the dosing chamber and preferably forms a part of the dosing head.

The air and ozone delivery ducts each include an inlet, which may be aligned with another part of the apparatus, which part is connected to the respective supplies of air and ozone.

The apparatus preferably includes a switch to switch on and off the supply of air and ozone.

### Reaction Chamber

According to another aspect of the invention there is provided an apparatus for producing and emitting hydroxyl radicals, the apparatus including an air supply, a supply of olefin and an ozone supply, means for introducing olefin into the air supply and mixing means for mixing ozone with the olefin/air mixture, and a reaction chamber, wherein ozone reacts with olefin in the reaction chamber, and wherein the reaction chamber is so shaped and dimensioned as to ensure that substantially all ozone supplied is reacted to form hydroxyl radicals prior to exiting the reaction chamber.

The dwell time of the ozone, air, olefin mixture in the reaction chamber is typically less than five seconds. The olefin ozone reaction time which produces the hydroxyl radicals is less than one second, but nevertheless temperature dependent.

Advantageously, the reaction chamber has at least one inlet and at least one outlet. The at least one inlet and at least one outlet are preferably disposed at the base and top of the reaction chamber respectively.

The reaction chamber may be provided with a cover, and preferably the cover includes the said at least one outlet. The cover may be in the form of a multi-legged element and preferably the multi-legged element has three legs. The legs of the said element may emanate from a central portion. Each leg may include part of a fastening arrangement, which arrangement fastens the cover to the reaction chamber.

In a preferred embodiment the reaction chamber mounts an actuator, the actuator operating the said switch. The actuator comprises a piston and cylinder arrangement and preferably the cylinder is formed in the cover. The piston may be biased by biasing means, such as a spring, such that the switch is in the off position. The piston may include a stationary reaction element and a movable element, the biasing means being disposed between the said elements. The reaction member may be held stationary by the dosing head. The reaction member may include a slot and a pin may pass through the slot and apertures in the movable element, such that the extent of movement of the movable element is determined by the length of the slot.

The reaction chamber is preferably part of a moulding which is removably attachable to another part of the apparatus. The other part of the apparatus is preferably a casing element.

Advantageously, the reaction chamber includes a housing for receiving a dosing head. The dosing head is preferably a push fit in the housing. An outlet of the dosing head may comprise an inlet of the reaction chamber.

The dosing head may include two outlets.

The cover may include dosing head engagement means to engage with the dosing head such that by lifting the cover the dosing head is lifted from the apparatus. Preferably the cover is movable between a first position in which lifting the cover does not lift the dosing head and a second position in which the engagement means engage with the dosing head. Advantageously, movement of the cover is accommodated by the resilience of the material from which it is made.

### Safety Device

According to another aspect of the invention there is provided an apparatus for producing and emitting hydroxyl radicals, the apparatus including an air supply, a supply of olefin and an ozone supply, means for introducing olefin into the air supply and mixing means for mixing ozone with the olefin/air mixture, further comprising a safety device, the said safety device causing the apparatus to be disabled in the event that substantial quantities of ozone are released from the apparatus.

The apparatus may include a reaction chamber.

Advantageously, the safety device includes an element formed of a material which fails in the presence of ozone. A suitable material would be one which exhibits micro-cracking in the presence of ozone. Such material may be rubber, for example natural or synthetic rubbers such as isoprenes, butadienes and polymers or derivatives thereof. In the event of ozone being released from the apparatus without reaction with olefin, for example in the case where the supply of olefin is exhausted, or malfunction of the apparatus, the material will fail indicating to the user that the apparatus is no longer in a safe condition. Such indication may consist of a graphic indication to a user of the apparatus. Preferably, upon failure of the material, the switch is disabled so that power may not be delivered to the ozone supply and/or air supply. An elongate element of such material may extend over an actuator of the apparatus.

### Consumable Element

According to another aspect of the invention there is provided a consumable element for an apparatus for producing and emitting hydroxyl radicals, the apparatus including an air supply, a supply of olefin and an ozone supply, means for introducing olefin into the air supply and mixing means for mixing ozone with the olefin/air mixture, wherein the mixing means is arranged such that ozone is mixed with the olefin air mixture at a period of time after introduction of olefin into the air supply, the consumable element comprising the supply of olefin, the means for introducing olefin into the air supply, the mixing means, and means for securing the consumable item in the apparatus.

Advantageously, the consumable element includes means for removing the consumable element from the apparatus. Preferably, the cover of the reaction chamber is movable to a position in which at least one part of the cover engages with another part of the consumable element such that the consumable element may be removed from the apparatus. The part of the cover may comprise at least one protrusion. In one embodiment the cover comprises at least one protrusion which engages with the dosing head.

With a cover in the form of a multi-legged element the user of the apparatus simply presses the cover towards the dosing head until the at least one protrusion is engaged with the dosing head, then grips the legs to lift the consumable element from the apparatus.

The outlets of the dosing head may form the inlets to the reaction chamber, with one outlet being larger than the other, and preferably the larger outlet being proximate the opening from the air/olefin mixing chamber to the ozone pathway.

The invention also provides a method of releasing open air factor into an environment comprising the step of actuating an apparatus according to the invention in the environment. Preferably the method includes the step of preventing unreacted ozone from being released into the environment, wherein unreacted ozone is detected and upon detection thereof the apparatus is disabled.

### BRIEF DESCRIPTION OF THE DRAWINGS

In the drawings, which illustrate preferred embodiments of the invention and are by way of example:

FIG. 1 is a schematic representation of a consumable item;

FIG. 2a is a cross-sectional elevation of a dosing head;

FIGS. 2b is a schematic representation of a cover part of the dosing head illustrated in FIG. 2a viewed from below;

FIG. 2c is a schematic representation of an inner part of the dosing head illustrated in FIG. 2a viewed from above;

FIG. 2d is a schematic representation of the inner part of the dosing head illustrated in FIG. 2c;

FIG. 2e is a is a schematic representation of the inner part illustrated in FIGS. 2c and 2d viewed from below;

FIG. 2f is a schematic representation of the assembled dosing head with the wick clamp inserted into the base thereof;

FIG. 2g is a schematic representation of an inner element of the dosing head;

FIG. 2h is a schematic representation of the dosing head viewed from above;

FIG. 3 is a schematic representation of apparatus according to the invention in assembled form;

FIG. 4 is a schematic representation of a component of the apparatus of the invention;

FIG. 5 is a schematic representation of a dosing head which is a component of the apparatus according to the invention;

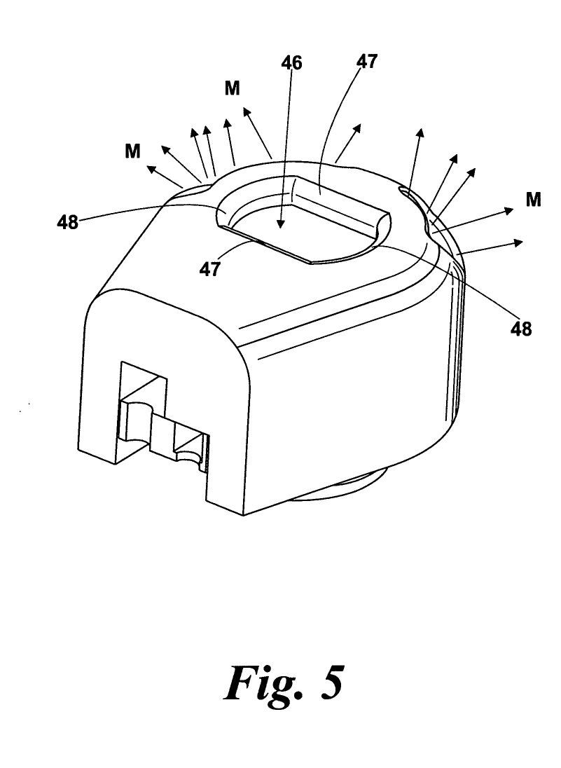

FIG. 6 is a schematic sectional representation of the internal components of the apparatus;

FIG. 7 is a schematic sectional representation of some of the internal components of the apparatus illustrated in FIG. 6 and viewed from the other side of the apparatus.

### DETAILED DESCRIPTION OF THE PREFERRED EMBODIMENTS

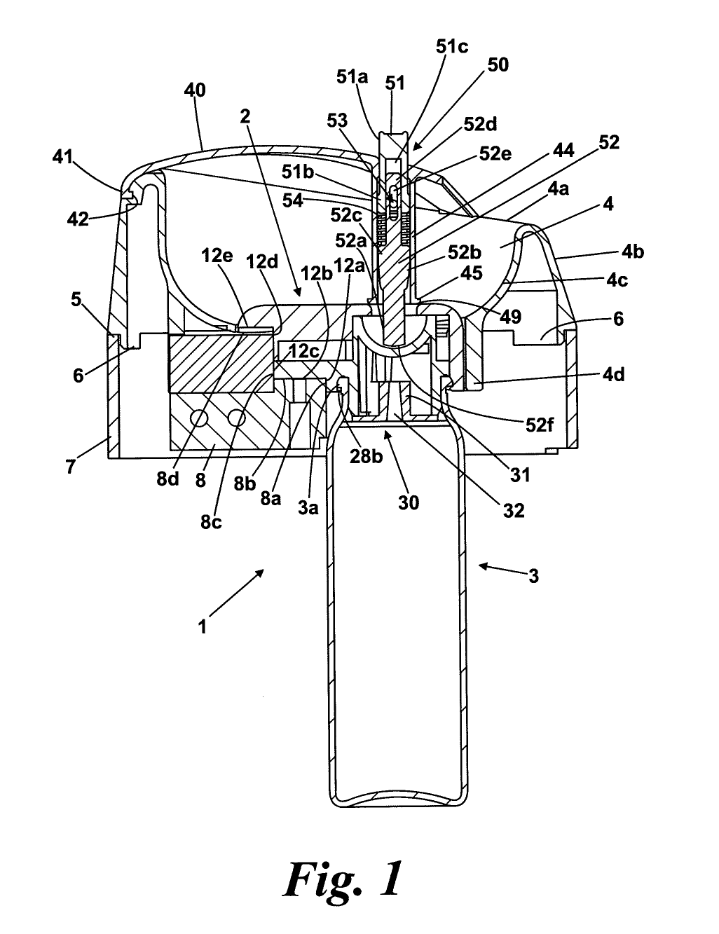

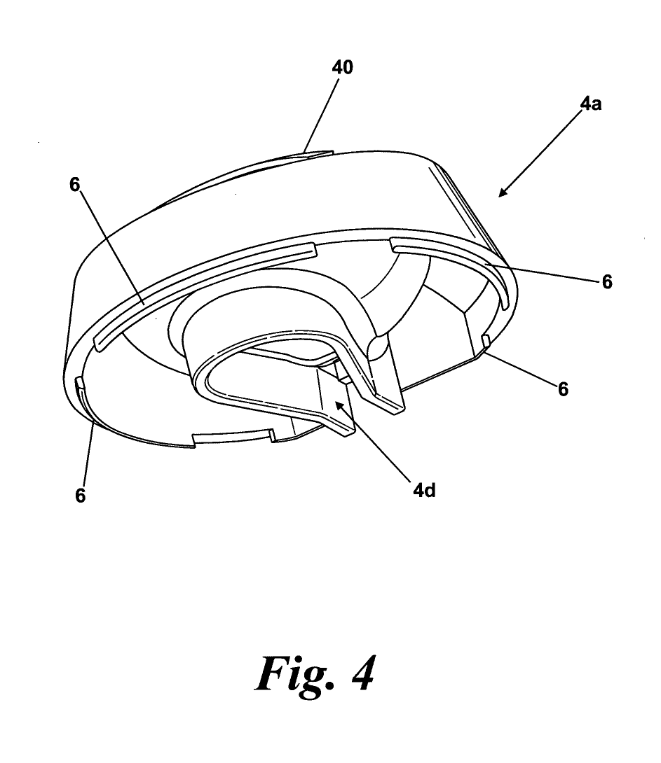

Referring now to FIG. 1, there is shown a consumable element 1 comprising a dosing head 2 and an olefin reservoir 3, the olefin being terpene in the present example, and a reaction chamber 4. The reaction chamber 4 is an integral part of a moulding 4a which includes an outer wall 4b providing a part of the casing of the device. The wall 4b has a bottom edge 5 from which extend a plurality of protrusions 6 which engage with the inner surface of a case 7, which encases a pipe block 8. The protrusions 6 are forced against the inner surface of the case 7 by virtue of the resilient nature of the moulding 4a. The moulding 4a and features thereof are illustrated in greater detail in FIG. 4.

The pipeblock 8 includes two outlet ports, which are aligned respectively with the inlet ports 21a and 21b of the dosing head 12.

In the dosing head 2 air is mixed with terpene and the terpene saturated air is mixed with ozone. The air/terpene/ozone mixture debouches into the reaction chamber 4 where it remains for a dwell time sufficient to ensure that a cascade of hydroxyl radicals is produced.

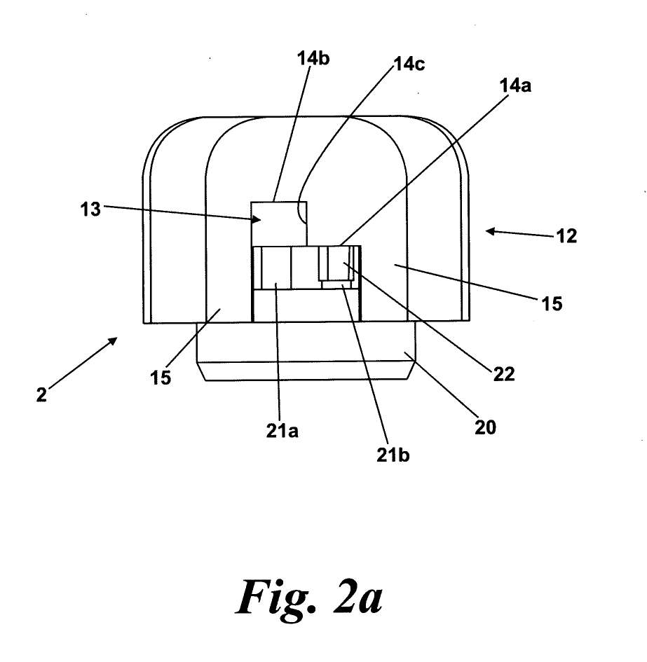

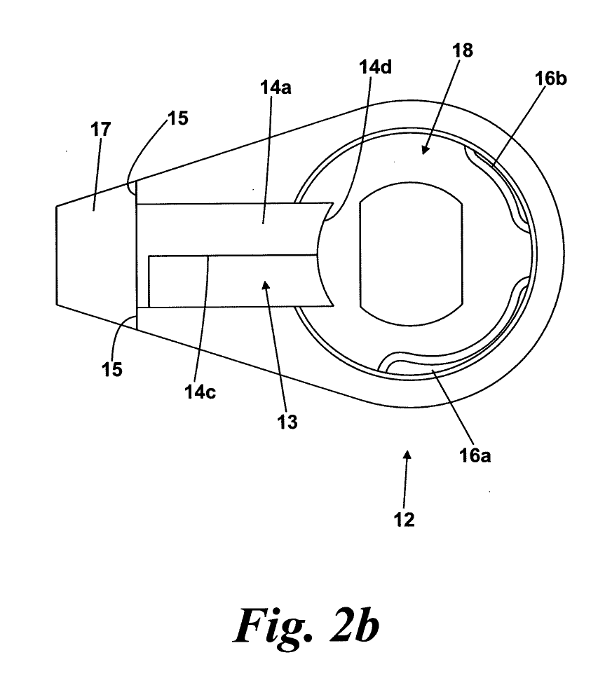

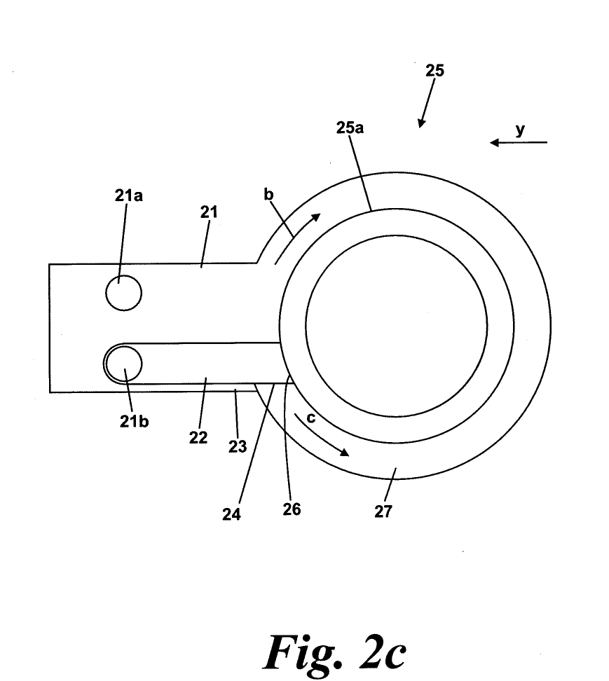

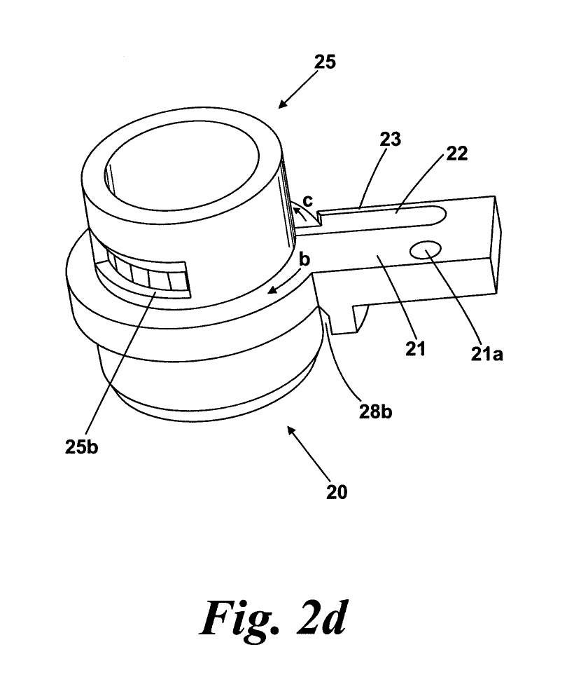

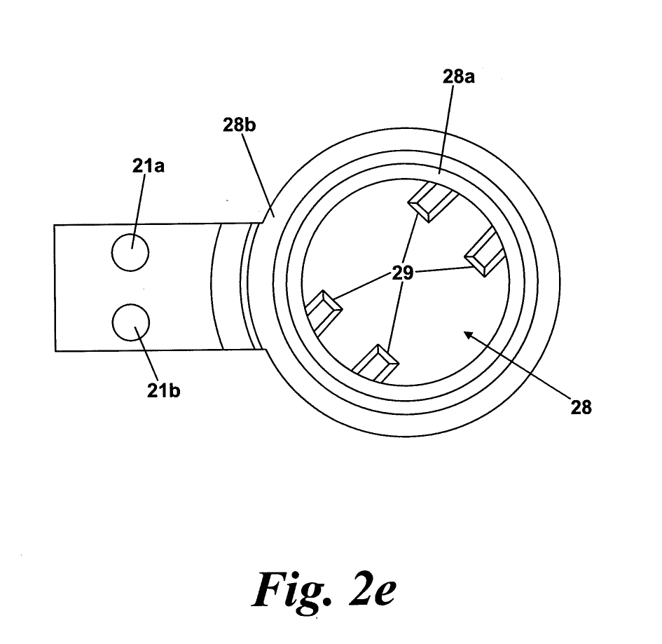

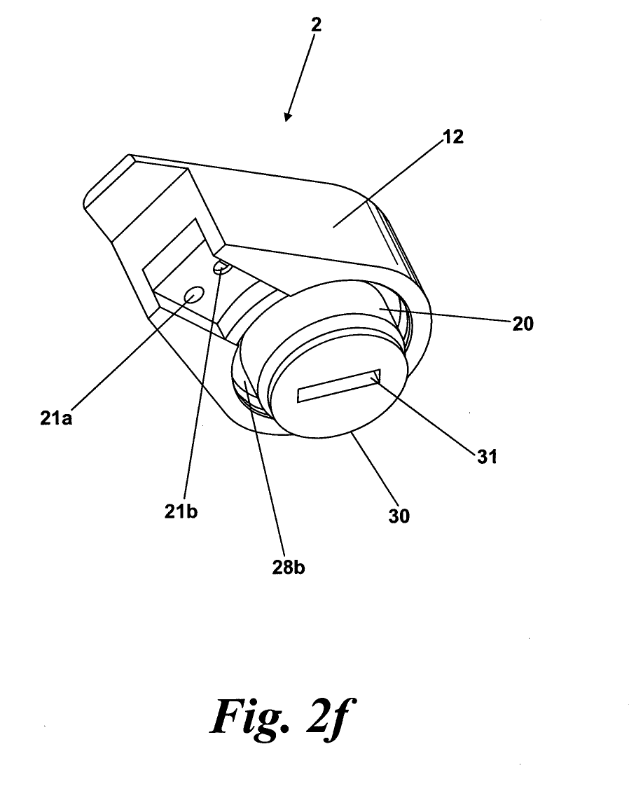

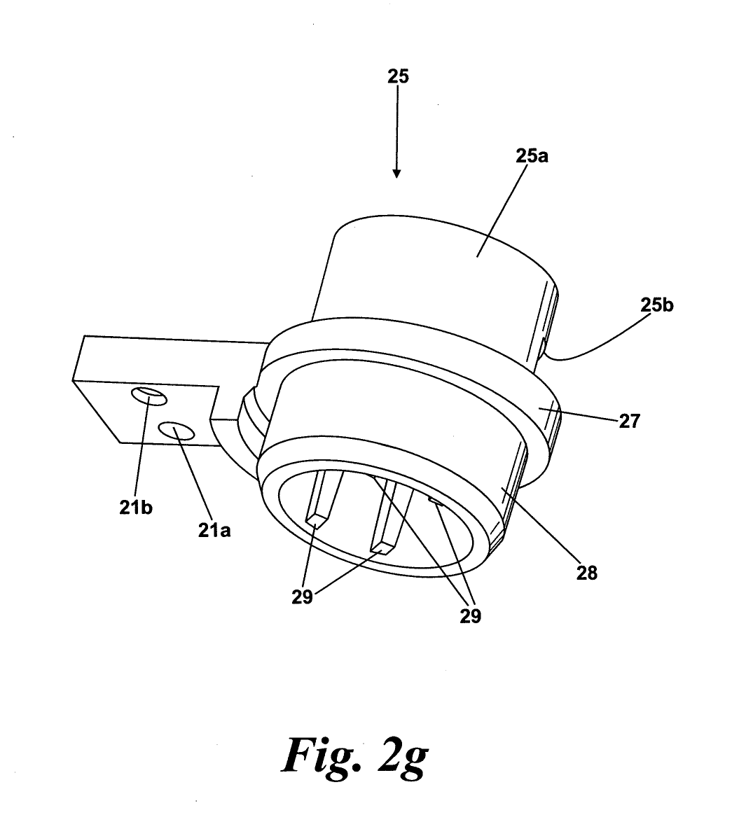

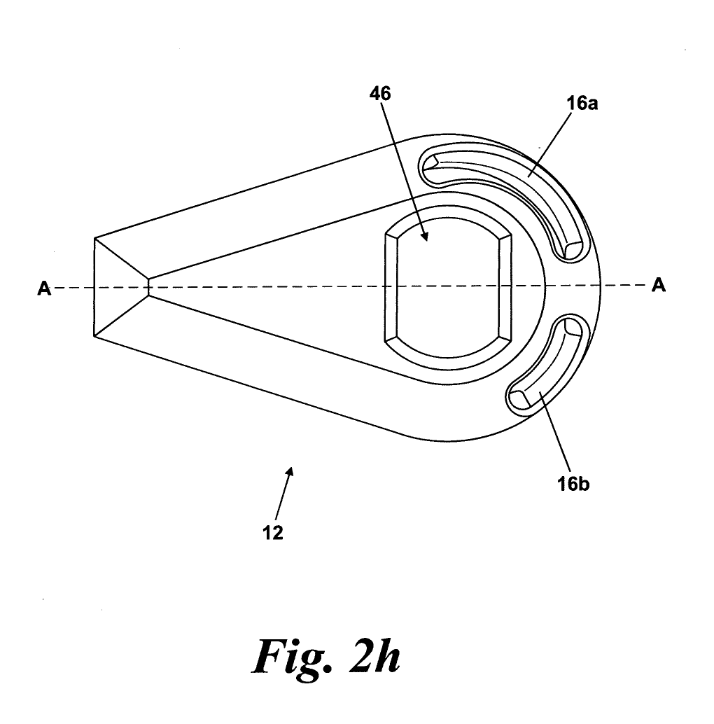

The dosing head 2 will now be described in greater detail with reference to FIGS. 2a to 2h. The dosing head 2 comprises an outer element 12 and an inner element 20. The inner element 20 includes two inlet ports 21a and 21b. When assembled, the inlet ports 21a and 21b are aligned with the outlet ports of the pipe block 8, one of which delivers a stream of air, the other delivering a stream of ozone. The inner element 20 includes a part of two channels 13 and 22, one which is arranged to deliver the aforementioned stream of air and the other the stream of ozone to the mixing chamber. The port 21a debouches into the channel 13 with the port 21b debouching into the channel 22.

As described above, the inner element 20 provides a part of each of the channels 13 and 22. The other part of each channel is provided by the outer element 12. Three sides of the channel 13 are formed by the outer element 12. The underside of the channel 13 is formed by the upper flat surface of the element 21. The channel 22 is formed on three sides by a channel 22 formed in the element 21, with the fourth side being formed by the underside of the surface 14a of the outer element 12. An end surface 14d of the element 14 is curved, the radius of curvature thereof, and the extent to which the element 14 protrudes into chamber 18 is such that the outer wall 25a of element 25 of the inner element 20 mates with the end surface 14d, thereby ensuring that ozone follows the path indicated by arrow b in FIG. 2c.

Referring specifically to FIG. 2c, the outer wall 23 of the element 21 ends at its intersection with the outer surface of a rib 27 which extends circumferentially around a cylindrical part 25 of the inner element 20. By terminating the wall 23 short of the wall 25a of the cylindrical part 25 an opening 24 is provided the purpose of which is described below. Directly in front of the end of the channel 22 is an opening 26 in the wall 25a into a chamber 28.

With the inner element 20 inserted into the outer element 12 a gallery is formed inside the outer element with the upper surface of the rib 27, the wall 25a and the wall 19 defining the said gallery. A pair of openings 16a and 16b is formed in the wall 19 of the outer element, one opening 16a and 16b being formed to each side of a centerline A-A.

It will be noted from FIG. 2h that the openings 16a and 16b are of unequal size, the opening 16a being larger than that of 16b. The opening 16a is substantially axially aligned with the opening 25b through which olefin/air mixture enters into the ozone stream.

As can be seen from FIG. 1, in the assembled device 1, the inner element 20 sits on top of the reservoir 3 with the wick 32 of the reservoir extending into the chamber 28 of the inner element. Upon actuation of the device, streams of ozone and air are delivered via the ports 21a, 21b to the channels 13 and 22. Ozone is delivered via channel 13 to the gallery following the path indicated by arrow b. Air is delivered via the channel 22 to the gallery following the path indicated by arrow c, and through the opening 26 into the chamber 28. The wick 32 is saturated with terpene and therefore as air passes through the opening 26 and into the chamber 28 the incoming air becomes loaded with terpene. The mixture of olefin and air exits the chamber 28 via an opening 25b. Thus the air/olefin mixture debouches tangentially into the radial path followed by the stream of ozone. As soon as the air/olefin mixture meets the incoming ozone a reaction begins to produce hydroxyl radicals. As will be apparent from the subsequent description, the reaction of ozone with olefin to produce hydroxyl radicals is not instantaneously complete.

The wall 23 ends before the wall 25a to leave an opening 24 which provides for a part of the air ingressing through port 21b to follow a radial path indicated by arrow c around the wall 25a. The air following path c meets the stream of ozone into which air/and terpene has been introduced in the region of the openings 16a and b. The air following path c enhances mixing of ozone with the olefin/air mixture, reduces the speed of the ozone/olefin/air mixture, and directs the resulting mixture to exit the gallery via the openings 16a and b. Openings 16a and b debouch into the reaction chamber 4 which is described in greater detail below.

The lower part of the chamber 28 is bounded by a wall 28a, and extending inwardly from the wall are two pairs of ribs 29.

Referring now to FIGS. 1 and 2f, a wick clamp 30 is inserted into the base of the chamber 28, the wick clamp 30 being held in position by the clamping element 31 being pushed into the openings provided by the respective pairs of ribs 29. The wick clamp 30 is attached to the reservoir 3, the upper wall of which comprises a lip 3a, which engages in a groove 28b to form a seal between the reservoir 3 and the dosing head 2.

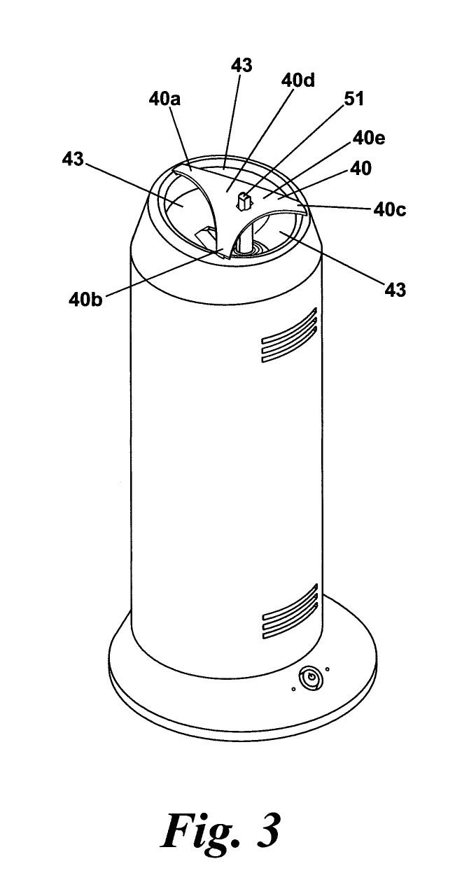

The reaction chamber 4 will now be described in greater detail with reference to FIGS. 1, 3 and 4. The reaction chamber 4 is part of a moulding 4a into which the dosing head 2 fits, the lower part of the moulding 4a including an aperture 4d which is so shaped and dimensioned as to receive the dosing head 12. The reaction chamber 4 is provided with a cover 40 which comprises three legs 40a, 40b and 40c and a central part 40d which includes an aperture 40e through which part of a switch actuator 50 protrudes. Each leg 40a-40c is provided with an inwardly extending element 41 which engages with a rebate 42 formed in the moulding 4a.

Emanating from the openings 16 of the dosing head 2 is a mixture of hydroxyl radicals resulting from the reaction of ozone with terpene loaded air, and unreacted ozone and terpene loaded air. The function of the reaction chamber 4 is to provide sufficient dwell time for the gases entering the chamber 4 to react together such that no unreacted ozone emanate from the openings in the top of the reaction chamber 4 between the legs 40a to 40c. The path followed by gases debouching from the openings 16 is determined by the shape of those openings. The size and shape of the openings 16 and their location in the base of the reaction chamber 4 means that gas debouching therefrom will impinge upon the curved wall 4c of the moulding 4 thereby initiating a swirling of the gaseous mixture in the reaction chamber 4. The cover 40 partially encloses the chamber 4 and causes the mixture to swirl in the reaction chamber 4. After a relatively short dwell time the ozone and terpene loaded air have reacted substantially completely and hydroxyl radicals exit the chamber 4 via the openings 43 in the cover 40.

In addition to forming a part of the reaction chamber 4, the cover 40 performs another function, namely to provide for the release and removal of the consumable element of the apparatus. The cover 40 includes a downwardly extending guide 44 the base of which comprises a protrusion 45 which extends around the base of the guide 44. As illustrated in FIG. 1 the apparatus is configured for use, with the protrusion 45 resting above an opening 46 in the outer element 12 of the dosing head 2.

To remove the consumable element from the apparatus the central part 40d of the cover 40 is depressed. This causes the lower part of guide 44 and hence protrusion 45 to move towards the opening 46 whereupon the chamfered outer surface 49 engages with correspondingly shaped surfaces of the opening 46. As the central part 40 of the cover is depressed further, the guide 44 is deformed, its sides being moved towards each other, and the protrusion 45 passes through the opening 46 in the outer element 12. When a force is exerted on the moulding 4 to pull it apart from the case of the apparatus, the dosing head and the reservoir 3 are lifted from the case with the moulding 4.

Arranged to slide in the guide 44 is the actuator 50 of the apparatus. The actuator 50 is made up of four components which slide inside the guide 44. Further, the actuator 50 provides a fail-safe mechanism which deactivates the apparatus in the event of unreacted ozone emanating from the reaction chamber 4.

The four components of the actuator 50 comprise a first element 51 in the form of a piston slidable within the guide 44, a second element 52, again in the form of a piston slidable within the guide 44, a pin 53 which connects the first element 51 to the second element 52 and a spring 54 which urges the first and second elements 51, 52 apart.

The first element 51 includes a first part 51a of a first cross-section and a second part 51b of a second cross-section, the first part 51a being so shaped and dimensioned as to be slidable within the opening 40e in the cover 40. The second part 51b is of a different and wider cross-section which is so shaped and dimensioned as to be slidable within the guide 44. The first element 51 includes a bore 51c which is so shaped and dimensioned as to receive slidably within it a part 52d of the second element 52. The second element 52 includes a first part 52a having a curved end 52f, the radius of curvature corresponding substantially to the radius of curvature of the element 25 of the dosing head 12. The element 52 includes a third part 52c which is so shaped and dimensioned as to slide within the guide 44, and a second part 52b extending between the first and second parts 52a, 52c. The shape of the part 52d provides for the protrusions 45 to move inward as they pass through the aperture 49. To assemble the actuator 50 the spring 54 is placed on the second element 52 to surround the part 52d thereof. The part 52d inserted into the bore 51c and the elements 51 and 52 are brought together so that holes in the first element 51 are aligned with the slot 52e in the second element and pin 53 is passed therethrough. With the actuator 50 so configured when a user exerts a pressure on the actuator 51 with a finger, the first element 51 moves axially downward within the guide 44 until the pin 53 engages with the base of the slot 52e and the second element 52 is forced axially downward within the guide 44. The position of the second element 52 is fixed as a result of its engagement with the inner element 25 of the dosing head. Depressing the first element 51 closes an electrical contact switch, which in turn actuates an air pump and an ozone generator. The air pump delivers air at a rate of 2.5 litres/minute with half the pumped air being delivered to the ozone generator and half being delivered directly to the dosing head 12. The ozone generator delivers an ozone rich gaseous stream to the dosing head 12.

The actuator 51a is covered with a flexible material which in the present example is rubber. The purpose of the flexible material is to provide part of a fail safe mechanism which disables the apparatus in the event that the supply of terpene from the reservoir 3 is exhausted. The purpose of disabling the apparatus when the terpene supply has been exhausted is to ensure that no ozone can be released into the atmosphere. Rubber develops micro-cracks in the presence of ozone.

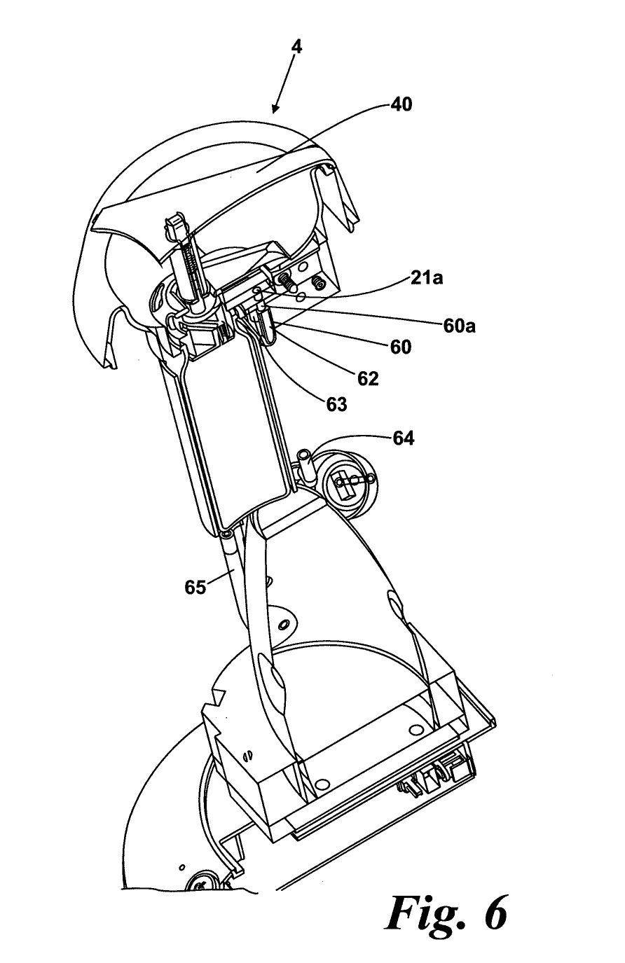

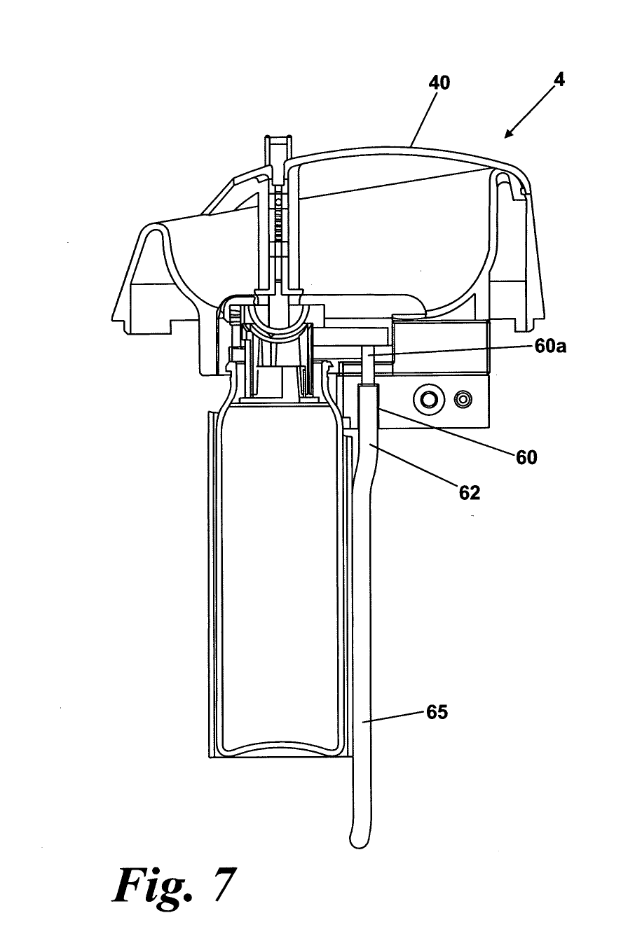

Referring now to FIGS. 1, 6 and 7, the pipe block 8 comprises bores 60, 60a and 61, 61a (not shown for the sake of clarity) which align with the ports 21a and 21b respectively. The bore 60a is a continuation of the bore 60, but of smaller diameter. The internal diameter of the bore 60 is determined to match the external diameter of the pipe 62 which is a push fit into the said bore 60. The internal diameter of the pipe 62 is substantially the same as the internal diameter of the bore 60a. The internal diameter of the bore 61 is determined to match the external diameter of the pipe 63 which is a push fit into the said bore 61. The internal diameter of the pipe 63 is substantially the same as the internal diameter of the bore 61. The pipe 62 connects to the pipe 65 which is in turn connected to the output of an ozone generator. The pipe 63 connects to the pipe 64 which is in turn connected to the output of an air pump.

Referring again to FIGS. 1, 6 and 7, the pipe block 8 provides reference surfaces 8a to 8d which ensure that the dosing head 12 is correctly positioned in the aperture 4d in the moulding 4a. The surfaces 8a, 8b and 8c engage with surfaces 12a, 12b and 12c of the dosing head 12. Further, the surface 8c engages with the surface 12d of the dosing head 12. Surface 12e sits above and is spaced apart from the surface 8d of the pipe block 8.

The consumable element, which comprises the reservoir 3, the dosing head 2, the reaction chamber 4, including the cover 40 are designed for single use and are discarded from the machine after one month of operation and are replaced by another consumable cartridge and cover for further use.

The consumable cartridge may be re-manufactured if so desired.

The apparatus of the invention provides for the efficient production of hydroxyl radicals, otherwise known as open air factor. This is achieved by introducing olefin into air and subsequently bringing together ozone and the mixture of olefin and air.

In another aspect of the invention a reaction chamber is provided into which ozone, olefin and air are introduced. The reaction chamber is so shaped and dimensioned that the dwell time of the mixture in the reaction chamber is such that substantially all the ozone entering the reaction chamber is reacted without leaving the chamber. Emanating from the chamber is a cascade of hydroxyl radicals.

Another aspect of the invention provides a consumable component comprising a reservoir of olefin, a dosing head, a reaction chamber and an actuator which provides for the efficient long term use of the apparatus. In one embodiment a cover element of the consumable component is configured such that moving the cover into a certain configuration allows the consumable component to be lifted from the apparatus.

Another aspect of the invention provides a safety mechanism for use with apparatus for producing and delivering hydroxyl radicals, the safety mechanism comprising a material which fails when exposed to ozone. The safety mechanism therefore ensures that substantive quantities of ozone may not be released into the atmosphere, for example in the event that the supply of olefin is exhausted or malfunction of the apparatus.

1-25. (canceled)

26. An apparatus for producing and delivering hydroxyl radicals, the apparatus including an air supply, a supply of olefin and an ozone supply, means for introducing olefin into the air supply and means for mixing ozone with the olefin/air mixture, wherein the mixing means is arranged such that ozone is mixed with the olefin/air mixture at a period of time after introduction of olefin into the air supply.

27. An apparatus according to claim 26, wherein the means for introducing olefin into the air supply and means for mixing ozone with the olefin/air mixture are comprised in a dosing head.

28. An apparatus according to claim 27, wherein the dosing head includes an inlet port connected to the air supply and an inlet port connected to the ozone supply, wherein the respective ports debouch into respective chambers, and wherein olefin is mixed with air in one of the respective chambers and that chamber debouches into the chamber through which ozone passes.

29. An apparatus according to claim 28, wherein the olefin supply is open to the air supply chamber downstream of the air supply inlet port and upstream of an opening to the ozone carrying chamber.

30. An apparatus according to claim 26, wherein the dosing head is adapted to deliver part of the air incoming from the air supply inlet port to olefin/air mixing chamber and part of the air incoming from said air supply inlet to the ozone carrying chamber.

31. An apparatus according to claim 30, wherein the part of the air incoming from said air supply inlet which is directed to the ozone carrying chamber flows in a direction opposite to the direction of flow of the ozone.

32. An apparatus according to claim 27, wherein the dosing head includes at least one outlet.

33. An apparatus according to claim 32, wherein the at least one outlet is located proximate the opening of the air/olefin mixing chamber into the ozone carrying chamber.

34. An apparatus according to claim 27, wherein the dosing head is provided with at least one surface which co-operates with at least one surface of another part of the apparatus, co-operation of the said surfaces ensuring the correct position of the dosing head in the apparatus

35. An apparatus according to claim 26, wherein the apparatus includes a reaction chamber.

36. An apparatus according to claim 35, wherein the reaction chamber is so shaped and dimensioned as to provide that the dwell time in the reaction chamber of a fluid mixture emanating from the dosing head is of such duration that the reaction of ozone and olefin in the fluid mixture entering the reaction chamber is complete.

37. An apparatus according to claim 35, further comprising a cover.

38. An apparatus according to claim 37, wherein the cover is in the form of a multi-legged element and at least one of the legs includes part of a fastening arrangement, which arrangement fastens the cover to the reaction chamber.

39. An apparatus according to claim 35, wherein the means for introducing olefin into the air supply and means for mixing ozone with the olefin/air mixture are comprised in a dosing head, and wherein the reaction chamber includes a housing for receiving the dosing head.

40. An apparatus according to claim 35, wherein the means for introducing olefin into the air supply and means for mixing ozone with the olefin/air mixture are comprised in a dosing head, and wherein an outlet of the dosing head comprises an inlet of the reaction chamber

41. An apparatus according to claim 37, wherein the cover includes dosing head engagement members adapted to engage with the dosing head such that lifting the cover with said members so engaged the dosing head may be lifted from the apparatus.

42. An apparatus according to claim 41, wherein the cover is movable between a first position in which dosing head engagement members are disengaged from the dosing head and a second position in which the dosing head engagement members are engaged with the dosing head.

43. An apparatus according to claim 42, wherein the cover is made of a resilient material and movement of said cover between the first and second positions is accommodated by the resilience of the material.

44. An apparatus according to claim 26, further including an actuator which is operable to power an air pump and an ozone generator, and wherein the actuator is mounted in the reaction chamber.

45. An apparatus according to claim 44, wherein the actuator is mounted in the cover of the reaction chamber.

46. A consumable element for an apparatus for producing and emitting hydroxyl radicals, the apparatus including an air supply, a supply of olefin and an ozone supply, means for introducing olefin into the air supply and mixing means for mixing ozone with the olefin/air mixture, wherein the mixing means is arranged such that ozone is mixed with the olefin air mixture at a period of time after introduction of olefin into the air supply, the consumable element comprising the supply of olefin, the means for introducing olefin into the air supply, the mixing means, and means for securing the consumable item in the apparatus.
